(19) United States
(12) Patent Application Publication
(10) Pub. No.: US 2026/0094703 A1
(43) Pub. Date: Apr. 2, 2026

(54) System for reprocessing medical devices

(71) Applicant: Bien-Air Holding SA, Biel/Bienne (CH)

(72) Inventors: Marco Gallina, Gletterens (CH); Davide Sarchi, Zürich (CH)

(21) Appl. No.: 19/110,356

(22) PCT Filed: Sep. 14, 2023
(86) PCT No.: PCT/EP2023/075365

§ 371 Date: Mar. 10, 2025

(30) Foreign Application Priority Data
Sep. 14, 2022 (EP) 22195689.9

(51) Int. Cl. G16H 40/40; G06K 7/10; G06K 19/07

(52) U.S. Cl. CPC G16H 40/40; G06K 7/10297; G06K 19/0723

(57) ABSTRACT

The present invention relates to the field of health and to the reprocessing of medical devices. In particular, the invention relates to a system and method for reprocessing medical devices. The system (1) for reprocessing medical devices (2) according to the invention comprises
an enclosure (3) suitable for the processing of medical devices (2), each of the medical devices (2) having at least one RFID tag (4);
at least one support (5) by which at least one medical device (2) to be reprocessed is intended to be suspended inside the enclosure (3);
at least one RFID tag reader (6);
at least one reprocessing ejector (7) arranged inside the enclosure (3);
a data management system (9) comprising processing software and a database relating to said medical devices and to reprocessing cycles;
   characterized in that said at least one RFID tag reader (6) is arranged at least partially in a wall of the enclosure (3) so as to transmit a signal and/or to receive in return the signal originating from at least one RFID tag (4) of at least one medical device (2) suspended inside the enclosure (3).

### FIELD OF THE INVENTION

The present invention concerns the field of health and the reprocessing of medical devices. More specifically, the invention relates to a system and a method for reprocessing medical devices.

### STATE OF THE ART

In the field of health, medical devices are subjected to reprocessing cycles such as sterilization, cleaning and/or disinfection.

These cycles can be traced using tags affixed to or engraved on these devices, such as an RFID tag or a barcode tag. A unique identifier for each medical device can then be detected by reading these tags, enabling reprocessing cycles for each medical device to be traced.

However, the reliability of reprocessing cycle traceability is not optimal, since tag detection typically takes place prior to reprocessing cycles, by means of tag readers placed outside the enclosure for reprocessing. The multiplication of tag reading steps outside the enclosure for reprocessing and the positioning of devices inside the enclosure thus increases the risk of error, making it impossible to guarantee the correct implementation of reprocessing cycles for each device.

Furthermore, in the event of a malfunction of the device for reprocessing, information concerning the validation of reprocessing cycles is not generated immediately and requires manual entry of the error codes issued by the device for reprocessing into a database.

Thus, there is a real need for a method for reprocessing medical devices whose reliability is improved by limiting the number of manual steps, with the help of a system enabling traceability and validation of reprocessing cycles to be managed efficiently and immediately. Patent document US2022047745 describes a system for reprocessing medical devices comprising a tag reader on the outside of the enclosure, and a support. The tag can be affixed to a part of the device located outside or inside the enclosure. The solution described in international application WO2008142300 from the same applicant discloses a system for reprocessing probes comprising an active part and a cable for connection to an imaging device. The active part is placed inside the enclosure volume for disinfection, while a suspension piece carries an RFID tag outside the enclosure and enables the cable to be suspended. Neither of these solutions deals with optimizing the transmission quality between medical device tags arranged inside an enclosure and a reader, and even less so for several medical devices to be processed simultaneously.

Patent document US2022211891 describes a system for reprocessing catheters, each of which can be attached to a tray. However, handling of the trays remains manual and does not allow any optimization of the position or orientation of the medical devices to be processed according to their type or size, in order to improve the reliability of transmission of traceability information in real time for each of them.

US20050148819 describes an endoscope cleaning system comprising an RFID tag, placed in a cleaning tank, on one part of which an RFID unit is arranged. Here again, no attempt is made to optimize transmission for this individual treatment of medical devices. The same reasoning applies to the solution disclosed in document US20090220377 concerning a device for cleaning and disinfecting an endoscope.

US20020161460 discloses yet another endoscope disinfection system comprising an RFID box arranged behind one of the walls of a cleaning chamber. The walls are made of non-metallic sheets (plastic or rubber) so that electromagnetic transmission of identification data can take place. The RFID tag can be attached directly to the endoscope placed in the cleaning chamber, and the endoscope can be attached to a support fixed in the cleaning chamber. However, here again there is no possibility of improving the reliability of transmission between tags linked respectively to a plurality of medical devices and a reader as part of batch processing.

### SUMMARY OF INVENTION

An object of the present invention is to provide a system making it possible to improve the reliability of the reprocessing of medical devices.

Another object of the present invention is to provide a system making it possible to ensure the traceability and the immediate, more reliable and more efficient validation of reprocessing cycles.

Thus, the invention relates to a system for reprocessing medical devices comprising
an enclosure suitable for the processing of medical devices, each of the medical devices having at least one RFID tag;
at least one support by which at least one medical device to be reprocessed is intended to be suspended inside the enclosure;
at least one RFID tag reader;
at least one reprocessing ejector arranged inside the enclosure;
an electronic control system; and
a data management system comprising processing software and a database relating to said medical devices and to reprocessing cycles;
characterized in that said at least one RFID tag reader is arranged at least partially in a wall of the enclosure so as to transmit a signal and/or to receive in return the signal originating from at least one RFID tag of at least one medical device suspended inside the enclosure,
at least one support comprising at least one movable part to enable both adjustable orientation of each medical device to be reprocessed relative to said RFID tag reader as well as adjustment of the position of said support within the enclosure,
said support, said at least one reader and said at least one ejector being connected to said electronic control system.

Medical devices within the scope of the present invention are devices and articles used in the medical, dental, surgical, pharmaceutical and/or veterinary fields. According to their design, medical devices within the meaning of the present invention comprise surfaces, a front part, a rear part, slots, grooves, joints and/or openings. They are made from materials such as steel, copper-nickel alloy and/or polymers resistant to reprocessing cycles, such as polyetheretherketone (PEEK) or polyamide-imide (PAI).

Preferably, medical devices are dental and/or surgical devices. These devices may comprise side surfaces, a rear part, and a front part which is the active part of the device. Preferably, medical devices are selected from among turbines, contra-angles and/or straight handpieces. Thus, within the framework of the invention, the position and orientation of the support can be adjusted individually for each medical device to be processed, and in particular according to its type, which determines its size within the enclosure, which is not the same, for example, between a straight handpiece and a contra-angle, as well as the positioning of the tags.

In the scope of the present invention, each medical device includes at least one tag. A tag associates a unique identifier with each medical device. Advantageously, the unique identifier is a unique numeric or alphanumeric code linked to a medical device, enabling clear and formal identification of a device and facilitating its traceability. This identifier can be detected when the tag is read by a suitable tag reader.

Preferably, each of the medical devices is fitted with two tags, so as to optimize detection reliability by ensuring that at least one of the two tags can be read, for example in the event of damage to one of them. The tags are typically RFID tags and/or barcode tags.

In the context of the present invention, the tags are RFID tags. Thus, according to the invention, the identification information comprises a unique identifier linked to each of the RFID tags of the medical devices. RFID tags are selected from passive and/or active RFID tags.

Passive RFID tags have no built-in power supply. The electromagnetic signal emitted by this type of RFID tag is created by a signal originating from the RFID reader. Advantageously, passive RFID tags are resistant to the very high temperatures, irradiation and/or chemical agents used during reprocessing cycles.

Active RFID tags have an internal power supply such as a battery. This type of tag is capable of transmitting an electromagnetic signal to the tag reader using wireless technology, and does not require an interrogation signal from the tag reader. Advantageously, the electromagnetic signal of an active RFID tag is more intense than that of a passive RFID tag.

Preferably, the tags are passive RFID tags, resistant to very high temperatures, irradiation and/or chemical agents, used during reprocessing cycles.

In addition, the tags are affixed and/or integrated on the side surfaces of the medical devices, by gluing, pressing or screwing, so as to increase the reliability of tag detection and reading

In the context of the present invention, the system comprises an enclosure suitable for reprocessing medical devices, whatever the complexity and diversity of the medical devices.

The enclosure consists of a top wall, a bottom wall, side walls and at least one door providing access to the interior of the enclosure designed to receive medical devices. Preferably, the enclosure comprises at least one removable door facilitating access to the interior of the enclosure. In addition, the closed enclosure is hermetic or watertight, so as to guarantee the correct implementation of reprocessing cycles, guarantee device sterilization and prevent the escape of bacteriologically contaminated fluids or gases outside the enclosure.

The various methods of cleaning and sterilizing medical devices use heat, water, chemical agents, various gases, and/or ionizing radiation emitted into the enclosure during the various reprocessing cycles. Thus, in the scope of the present invention, the system comprises at least one reprocessing ejector arranged inside the enclosure.

The reprocessing ejector is configured to eject steam, a chemical agent, water, gases, ionizing radiation and/or compressed air, in order to clean and/or sterilize medical devices.

Inside the enclosure, at least one support is designed to suspend at least one medical device to be reprocessed. Preferably, each support is designed to suspend one and only one medical device to be reprocessed. Advantageously, suspending the medical devices enables a greater proportion of the devices'surfaces, cavities and/or channels to be reprocessed effectively, in contrast to devices arranged in cassettes or on trays.

According to the present invention, the supports comprise fastening means adapted to hold at least one medical device in suspension. Preferably, the fastening means are selected from among a clamp, a screw, a clip or a magnetic adhesive. These fastening means are adapted to hold the devices to be reprocessed in suspension at the level of certain parts of the device so as to ensure complete reprocessing of the parts intended for treating patients.

In the context of the present invention, it is conceivable that at least one ejector may be arranged inside a support. Preferably, when the support has a tubular shape, at least one ejector can be arranged inside the tubular shape of the support. Advantageously, the substances produced by reprocessing cycles are thus ejected inside the medical device, at openings such as cavities or internal channels, rather than onto the medical device's external surfaces.

In the particular case of dental and/or surgical devices, the fastening means are adapted to suspend the devices to be reprocessed at the rear part, ensuring complete reprocessing of the front part, which is the active part intended for treating patients.

It is conceivable that the fastening means also comprise an envelope designed to receive a suspended medical device. Advantageously, the envelope suspended by means of the fastening means enables complete reprocessing of the medical device. Preferably, the envelope is a sterilization pouch.

It is also possible for each envelope to include an RFID tag. Thus, by combining the device identifier with that of the envelope, the traceability of the envelope containing a device is ensured right up to its use. In particular, the system makes it possible to trace the sterility period of the device in its envelope. This is particularly advantageous as sterility is often limited to a few days. Additional sterilization/decontamination is recommended if such a period is exceeded.

In the context of the present invention, the supports are attached to a wall of the enclosure. Preferably, the supports are attached to the top wall of the enclosure. The position of the supports inside the enclosure is adjustable in such a way as to adjust the distance between the medical devices according to their size, avoid any contact between them and optimize the orientation of the RFID tags of each of the medical devices.

Advantageously, each medical device is kept suspended inside the enclosure without direct contact with other devices or elements inside the enclosure, such as walls or metal surfaces, thus limiting damage to the tags affixed and/or integrated on the side surfaces of the medical devices.

According to the present invention, the supports comprise at least one movable part to enable adjustable orientation of the medical device, so as to optimize the orientation of the RFID tags for each of the medical devices.

In the context of the present invention, RFID tags affixed and/or embedded on the surfaces of medical devices are read by RFID tag readers. Thus, the system comprises at least one RFID tag reader, arranged at least partially in a wall of the enclosure so as to emit a signal and/or receive in return the signal coming from the RFID tags of the medical devices suspended inside the enclosure.

Advantageously, the arrangement of the RFID tag reader at least partially within a wall of the enclosure means that the signal from the RFID tags of the medical devices can only be received back inside the enclosure. In addition, this arrangement protects the RFID tag reader from substances projected during reprocessing cycles, while minimizing the reader's footprint inside the enclosure.

It is conceivable that one part of the RFID tag reader, for example the part containing the electrical/electronic circuits, is arranged so as to be totally integrated into the wall, while another part is arranged so as to extend into the interior of the enclosure volume.

In a particular aspect of the invention, the invention concerns a system wherein said at least one RFID tag reader comprises guiding means arranged to guide a part of the RFID tag reader inside the enclosure so as to acquire the identification information of each of the medical devices. The guiding means are, for example, an articulated arm adapted to direct the reading head of the RFID tag reader close to the RFID tags of the medical devices. It is advantageous to be able to adjust the position of part of the RFID tag reader inside the enclosure according to the type and size of the medical device to be reprocessed. This ensures optimum wireless transmission quality between the RFID tag and the RFID reader. This minimizes the risk of errors during the procedure for reprocessing and transmission of identification information. The reliability of tracking is thus maximized. What's more, the articulated arm is designed to be folded back during reprocessing cycles so as to protect the reading head of the RFID tag reader from substances released during reprocessing cycles.

According to another particular aspect, the invention concerns a system for reprocessing medical devices wherein said at least one RFID tag reader is arranged completely in a wall of the enclosure so as to emit a signal and/or receive in return the signal coming from the RFID tags of the medical devices suspended inside the enclosure. Advantageously, this arrangement protects all parts of the RFID tag reader from substances released during reprocessing cycles, while minimizing the reader's footprint inside the enclosure. In particular, the electrical/electronic circuits and the part comprising the reading head are protected while remaining functional.

Advantageously, the partial or total arrangement of the tag reader in a wall maximizes the angle or region of detection of the RFID tags positioned on the devices to be reprocessed, so as to cover a large volume or even the entire volume of the enclosure.

In the context of the present invention, it is conceivable that RFID tag readers are formed by extrusion or overmolding in at least one wall of the enclosure. As the tag reader is thus integral with the wall, the system does not require any assembly steps at the time of use, and can be easily implemented.

In addition, RFID tag readers are arranged to acquire identification information for each medical device before, during and after reprocessing cycles of said medical devices held in suspension inside the enclosure. In this way, the system enables the status of the medical devices to be known immediately before the start of a reprocessing cycle, during and just after the end of said reprocessing cycle. In this way, the system ensures that the status of medical devices can be monitored, and enables their treatment status—i.e. whether they need to be reprocessed or have actually been reprocessed—to be validated after each reprocessing cycle.

In addition, the system according to the invention comprises at least one metal plate arranged to be movably positioned outside the enclosure to prevent RFID tags from being read outside the enclosure. The arrangement of at least one metal plate on one or more sides of the enclosure is adaptable to the location of the system. Preferably, the system comprises at least two, three, four, five or six metal plates. The metal of the plates is chosen from conductors such as aluminum and stainless steels and/or from plastic materials (PET) metallized with electrical or magnetic dopants (such as aluminum, fluorine, or tin), so as to prevent RFID tags from being read outside the enclosure.

Advantageously, the traceability and validation of reprocessing cycles is ensured immediately, in the most reliable and efficient way, by an electronic control system linked to a management system preferably comprising processing software and a database managing identifiers individually associated with devices, for example via RFID tags. In this way, processing statuses can be updated in real time and individually for each device as different reprocessing cycles take place. Immediate information transmission thus optimizes the reliability of the system for reprocessing and of the method implemented by this system.

The electronic control system comprises a control unit able to
control the RFID tag reader or readers, and the reprocessing ejector or ejectors;
control the support or supports;
collect identification data acquired by the RFID tag reader or readers;
transfer the identification data acquired by the RFID tag reader or readers to the data management system; and
transfer the data relating to reprocessing cycles to the data management system.

According to the present invention, the identification data acquired by the RFID tag readers are the unique identifiers of medical devices and/or envelopes.

During a reprocessing cycle, error codes can be issued by the electronic control system when a complete reprocessing cycle has not been completed, for example in the case of a failure or loss of sealing of the enclosure. In this way, reprocessing cycle information includes the number of cycles, the type of reprocessing cycles carried out, and the error codes generated during the reprocessing cycles.

In the scope of the present invention, the electronic control system comprises a control unit capable of transferring identification information and reprocessing cycle information to the data management system, whose role is to ensure and monitor, in particular via processing software interacting with a database, the smooth running of each of the reprocessing cycles, at the same time updating the processing status of the medical devices. In this way, data from the data management system includes medical device identifiers, envelope identifiers, medical device references, the number and type of reprocessing cycles carried out for each medical device, and any error codes generated during reprocessing cycles.

The present invention also relates to a method for reprocessing medical devices carried out with the aid of a system such as that defined in the foregoing.

One object of the present invention is to provide a method for reprocessing medical devices making it possible to improve the reliability of the reprocessing of medical devices.

Another object of the present invention is to provide a method for reprocessing medical devices making it possible to ensure the traceability and the validation of reprocessing cycles in a more reliable and efficient way.

Thus, the invention relates to a method for reprocessing medical devices with the aid of a system for reprocessing comprising the following steps:
i. equip at least one medical device to be reprocessed with at least one RFID tag;
ii. position inside an enclosure for reprocessing said at least one medical device comprising at least one RFID tag and hold each said at least one medical device in suspension with the aid of a support;
iii. close the enclosure;
iv. adjust the position of at least one support in the enclosure and the orientation of said support relative to at least one RFID tag reader for each medical device to be reprocessed;
v. activate said at least one RFID tag reader in order to collect identification data from each of the medical devices before carrying out a reprocessing cycle;
vi. activate said at least one reprocessing ejector to perform at least one reprocessing cycle;
vii. collect the reprocessing cycle data;
viii. activate said at least one RFID tag reader so as to collect identification data from at least one medical device after completion of the reprocessing cycle; and
ix. transfer the identification data acquired by said at least one RFID tag reader and the data on the reprocessing cycles to a data management system;
x. update the data relating to each medical device in a database of said data management system after each reprocessing cycle.

In the method for reprocessing medical devices of the invention, activation of one or more reprocessing ejectors enables one or more reprocessing cycles selected from among cleaning, disinfection, lubrication and/or sterilization to be carried out.

It is conceivable that at least one of the medical devices to be reprocessed is placed inside an envelope held suspended with the aid of a support inside the enclosure. Advantageously, the envelope suspended with the of a support enables complete reprocessing of the medical device. Preferably, the envelope is a sterilization pouch.

It is also conceivable for each envelope to include an RFID tag. Thus, by combining the device identifier with that of the envelope, the traceability of the envelope containing a device is ensured right up to its use. In particular, the method makes it possible to trace the period during which the device in its envelope is sterile. This is particularly advantageous in the case where sterility is limited to a few days.

According to one implementation of the method, the RFID tag readers are activated continuously throughout the reprocessing cycles.

Thus, the identification information for each medical device and the reprocessing cycle information are transmitted by the electronic control system to the data management system so as to acquire traceability information for each medical device. This traceability information consists, for example, of the number of reprocessing cycles to which each device has already been subjected, and when, so as to determine an instantaneous status for each medical device indicating in particular whether reprocessing is due, but not yet carried out; whether reprocessing is in progress—in the event that both the RFID reader and the RFID identifiers remain active throughout a reprocessing cycle—or whether reprocessing has just been correctly carried out. Advantageously, this traceability information is acquired immediately by the data management system.

In addition, a further validation step of the method for reprocessing can be envisaged, in which the data management system is configured to validate the reprocessing of each of the medical devices on the basis of the traceability information.

### BRIEF DESCRIPTION OF THE FIGURES

The particular features and advantages of the present invention will appear in greater detail in the context of the following description with an example of implementation given by way of illustration and in a non-limiting manner with reference to the annexed drawings in which:

FIG. 1 is a schematic representation of a system for reprocessing medical devices according to an embodiment of the invention.

FIG. 2 is a schematic representation of a system for reprocessing medical devices according to another embodiment of the invention.

FIG. 3 is a schematic representation of the interior of the enclosure of a system for reprocessing medical devices in which the supports have a moving part and allow an adjustable orientation of the medical device in relation to the position of the RFID tag reader.

### DETAILED DESCRIPTION OF THE INVENTION

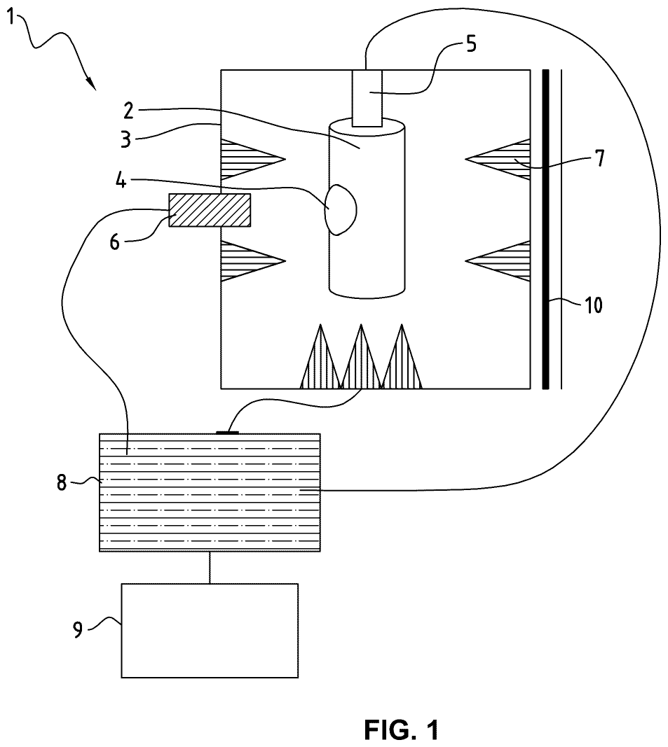

FIG. 1 schematically and functionally illustrates a system for reprocessing 1 medical devices 2 according to a preferred embodiment of the present invention. The medical devices 2 are each fitted with an RFID tag 4, and the system comprises an enclosure 3, a metal plate 10, a plurality of supports 5 of which only one is visible, an RFID tag reader 6, a plurality of reprocessing ejectors 7, an electronic control system 8 and a data management system 9, which includes processing software, i.e. computer software comprising a program for executing various reprocessing cycles. The latter interacts with a regularly updated database to indicate the respective instantaneous reprocessing status of the medical devices 2, before, during and after each reprocessing cycle.

The enclosure 3 comprises right and left side walls, a rear side wall (not shown), a top wall, a bottom wall, and a door (not shown) allowing access to the interior of the enclosure 3.

In certain embodiments, the enclosure 3 comprises at least one removable door facilitating access to the interior of the enclosure 3.

The enclosure 3 delimits a volume inside the enclosure 3 in which the reprocessing cycles of medical devices 2 are carried out. When the door is closed, the enclosure 3 delimits a hermetically sealed volume in such a way as to ensure the correct implementation of the reprocessing cycles and the sterility of the medical devices 2.

In the embodiment illustrated in FIG. 1, the system for reprocessing 1 comprises a support 5 fixed to the upper wall of the enclosure 3.

In other embodiments, a plurality of supports can be provided and attached to the top wall of the enclosure 3. The fixing of the supports 5 to the top wall of the enclosure 3 can be adjusted according to the overall dimensions of the medical devices 2 in relation to one another.

According to other embodiments, the support 5 can comprise a movable part to allow an adjustable orientation of the medical device 2.

The support 5 is adapted to hold in suspension preferably one medical device 2 at a time to be reprocessed inside the enclosure 3 by means of fastening means not shown in FIG. 1, such as a clamp, screw, clip or magnetic adhesive. In other alternative embodiments, a support 5 can hold a plurality of medical devices (2) in suspension at the same time.

The holding in suspension of medical devices as illustrated in FIG. 1 makes possible an efficient reprocessing of a greater part of the surfaces and/or cavities of the medical devices 2.

In the embodiment illustrated in FIG. 1, the system for reprocessing 1 comprises a plurality of reprocessing ejectors 7 arranged inside the enclosure 3 so as to carry out the reprocessing cycles of the medical devices 2 in the volume delimited by the enclosure 3. In other embodiments, a single ejector may be provided inside the enclosure 3. An ejector 7 can be configured to perform different reprocessing cycles, such as ejecting steam, a chemical agent, water, gas, ionizing radiation, or compressed air, in order to clean and/or sterilize medical devices 2.

In the embodiment illustrated in FIG. 1, without being limiting, the support 5 has a tubular shape. It is conceivable that at least one ejector 7 is arranged inside the tubular shape of the support 5. Advantageously, the reprocessing substances are ejected inside the medical device, at the level of openings in the medical device 2, such as cavities or internal channels.

In the embodiment illustrated in FIG. 1, the system for reprocessing 1 comprises an RFID tag reader 6 partially arranged in a wall of the enclosure 3 so as to emit a signal and/or receive in return the signal from the RFID tags 4 of the medical devices 2 suspended inside the enclosure 3.

According to this embodiment, the RFID tag reader 6 is arranged partially in one of the lateral walls of the enclosure 3.

In another embodiment, the system can comprise a plurality of RFID tag readers 6 partially arranged in one or more walls of the enclosure 3 so as to emit a signal and/or receive in return the signal from the RFID tags 4 of the medical devices 2 suspended inside the enclosure 3.

In this way, the RFID tag reader 6 can receive in return the signal from the RFID tags 4 of the medical devices 2 located only inside the enclosure and not outside the enclosure 3. In addition, this arrangement protects the RFID tag reader 6 from substances projected during reprocessing cycles, while minimizing the space required by the reader 6 inside the enclosure 3.

In the embodiment illustrated in FIG. 1, the part comprising the electrical/electronic circuits of the RFID tag reader 6 is arranged outside the enclosure 3, while the part comprising the reading head of the RFID tag reader 6 extends through one of the side walls of the enclosure 3 towards the interior of the volume of the enclosure 3 so as to emit a signal and/or receive in return the signal coming from the RFID tags 4 of the medical devices 2 suspended inside the enclosure 3.

In another embodiment, the part comprising the electrical/electronic circuits of the RFID tag reader 6 is fully integrated into one of the side walls of the enclosure 3, while the part comprising the reading head of the RFID tag reader 6 extends through this wall into the volume of the enclosure 3.

In another embodiment, the RFID tag reader 6 further comprises guide means arranged in one of the side walls of the enclosure 3 and adapted to direct part of the RFID tag reader 6 or the reading head of the RFID tag reader 6 close to the RFID tags 4 of the medical devices 2. These guiding means are, for example, an articulated arm. Advantageously, the articulated arm is adapted to be folded back at the time of reprocessing cycles so as to protect the reading head of the RFID tag reader 6 from substances projected during reprocessing cycles.

In another embodiment, the system for reprocessing 1 comprises an RFID tag reader 6 arranged entirely in one wall of the enclosure 3 so as to emit a signal and/or receive in return the signal from the RFID tags 4 of the medical devices 2 suspended inside the enclosure 3.

In the embodiment illustrated in FIG. 1, the system for reprocessing 1 comprises an electronic control system 8 including a control unit able to
control the RFID tag reader 6, and the ejectors 7 for reprocessing;
control the support 5;
collect the identification data acquired by the RFID tag reader 6;
transfer the identification data acquired by the RFID tag reader 6 to the data management system 9; and
transfer the reprocessing cycle information to the data management system 9.

The support 5, the RFID tag reader 6, and the reprocessing ejectors 7 are connected to the electronic control system 8 by a wired connection. In another embodiment, the support 5, the RFID tag reader 6, and the reprocessing ejectors 7 are connected to the electronic control system 8 by a wireless connection.

The electronic control system 8 is connected to the data management system 9 by a wired connection. According to another embodiment, the electronic control system 8 is connected to the data management system 9 by a wireless connection.

In the embodiment illustrated in FIG. 1, the system for reprocessing 1 comprises a metal plate 10 arranged outside the enclosure to prevent RFID tags from being read outside the enclosure.

According to another embodiment, the system for reprocessing 1 can comprise a plurality of metal plates 10.

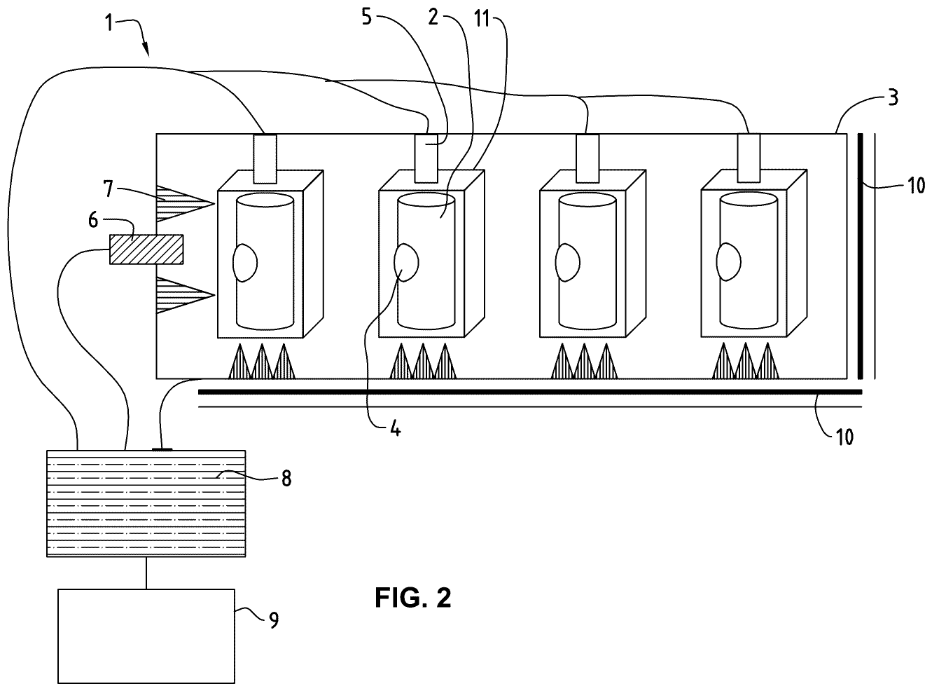

FIG. 2 schematically illustrates a system for reprocessing (1) of medical devices 2 comprising an enclosure 3, two metal plates 10, a plurality of supports 5, an RFID tag reader 6, a plurality of reprocessing ejectors 7, an electronic control system 8 and a data management system 9.

In the embodiment shown in FIG. 2, medical devices 2 each fitted with an RFID tag 4 are respectively arranged in envelopes 11 suspended inside the enclosure 3 with the aid of support fastening means 5.

Advantageously, the envelopes 11 suspended with the aid of the support fastening means 5 make possible a complete reprocessing of the medical devices 2.

According to another embodiment, the medical devices 2 can be each fitted with two RFID tags 4.

According to another embodiment, the envelopes 11 can be each fitted with an RFID tag.

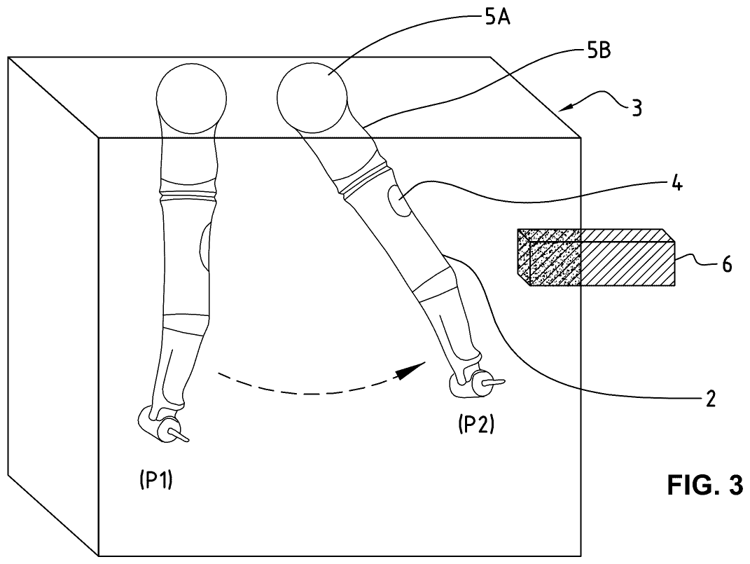

FIG. 3 is a schematic representation of the interior of the enclosure 3 of a system for reprocessing 1 medical devices 2 fitted with an RFID tag 4.

Supports 5 are fixed to the upper wall of the enclosure 3 and comprise a first attachment part 5A, mounted on said wall corresponding to the upper part, i.e. the ceiling of the enclosure, and a second movable part 5B, comprising fixing means adapted to keep the medical devices 2 suspended inside the enclosure 3.

The position of the supports 5 inside the enclosure 3 can be adjusted by means of the first attachment part 5A so as to adjust the distance between the medical devices 2 according to their size, avoid any contact between them and optimize the orientation of the RFID tags 4 of each of the medical devices 2.

In a particular, non-limiting example, the devices can be held either in a first, vertical position P1, or moved to a second, inclined position P2, according to the needs. The arrow indicates the transition from the first position P1 to the second position P2, which may be at a predetermined angle to the vertical, along two orthogonal axes (FIG. 3).

According to the variant illustrated in FIG. 3, the first attachment part 5A is in the form of a spherical ball joint allowing any positioning of the second mobile part 5B to which the medical device 2 is fixed inside the enclosure thanks to two rotational degrees of freedom in a spherical coordinate system θ, φ.

The positioning of the medical device 2 around the longitudinal axis along which the second movable part 5B extends has changed between the first position P1 and the second position P2, corresponding to a third degree of rotational freedom, precisely around this longitudinal axis. This latter adjustment can be made either by freely positioning the medical device 2 relative to the second movable part 5B of the support 5 when it is attached, or by conferring a third degree of rotational freedom on the ball-and-socket joint enabling this adjustment to be made.

Whichever variant is chosen, the three-degree-of-freedom rotational adjustment aims on the one hand to bring the RFID tags 4 as close as possible to the reader 6, but also to adjust the orientation of the RFID tags 4 in relation to the reader 6, so as to ensure, in a reliable way, the efficient transmission via the air interface.

The methods described in FIGS. 1 to 3 are equally suitable for use with passive and/or active RFID tags.

The method for reprocessing medical devices 2 according to the present invention can be achieved with the aid of a system for reprocessing 1 such as described or illustrated in the foregoing.

In a first step, it is necessary to equip each of the medical devices 2 to be reprocessed with at least one RFID tag 4. The RFID tags 4 can be affixed and/or integrated on the side surfaces of the medical devices 2 by methods such as gluing, pressing or screwing.

The medical devices 2 fitted with at least one RFID tag 4 are then positioned inside an enclosure 3 for reprocessing and held in suspension with the aid of supports 5 comprising fastening means such as a clamp, screw, clip or magnetic adhesive. In a particular embodiment of the method, each of the medical devices 2 to be reprocessed is placed inside an envelope 11 held in suspension with the aid of the fastening means of a support 5 inside the enclosure 3.

The door of the enclosure 3 is closed so as to ensure the tightness necessary for the proper implementation of reprocessing cycles and to ensure the sterility of the medical devices 2.

The RFID tag reader 6 is activated by the electronic control system 8 so as to collect identification data from each of the medical devices 2 held in suspension inside the enclosure 3 before performing a reprocessing cycle. The identification data, comprising a unique identifier linked to each of the medical devices 2, is then detected by reading the RFID tags 4 affixed/integrated on the surfaces of the medical devices 2.

The reprocessing ejectors 7 are then activated by the electronic control system 8 to perform a reprocessing cycle such as the ejection of steam, a chemical agent, water, gas, ionizing radiation or compressed air, in order to clean and/or sterilize the medical devices 2.

All the data connected with the reprocessing cycles such as the number, the type of reprocessing cycle and the error codes are collected by the electronic control system 8.

The following step involves activation of the RFID tag reader 6 by the electronic control system 8 to collect identification data from each of the medical devices 2 after the reprocessing cycle has been completed.

During this method, all identification data acquired by RFID tag readers 6 and reprocessing cycle data are immediately transferred by the electronic control system 8 to the data management system 9. This information makes it possible to ensure the traceability of the reprocessing of each individual medical device in an optimal way.

In addition, the method includes a step in which the data management system 9 can be configured to validate or not the reprocessing of each medical device 2 on the basis of traceability information. Any error code issued during reprocessing cycles will immediately cause the data management system not to validate the reprocessing of the medical devices.

1. System for reprocessing medical devices comprising:
an enclosure suitable for processing medical devices, each of the medical devices having an RFID tag;
a support by which a medical device to be reprocessed is to be suspended inside the enclosure;
an RFID tag reader;
a reprocessing ejector arranged inside the enclosure;
an electronic control system; and
a data management system comprising processing software and a database relating to said medical devices and to reprocessing cycles;
wherein said RFID tag reader is arranged at least partially in a wall of the enclosure so as to transmit a signal and/or to receive in return a signal originating from at least one RFID tag of at least one medical device suspended inside the enclosure,
said support comprising a movable part to enable both adjustable orientation of each medical device to be reprocessed relative to said RFID tag reader, and adjustment of a position of said support within the enclosure,
said support, said RFID tag reader and said reprocessing ejector being connected to said electronic control system.

2. System according to claim 1, wherein the electronic control system comprises a control unit able to:
control a plurality of said RFID tag reader, and a plurality of said reprocessing ejectors;
individually control respective positions of a plurality of said supports inside the enclosure and an orientation of the movable part of each of said supports;
collect identification data acquired by said plurality of RFID tag readers;
transfer the identification data acquired by said RFID tag readers to the data management system; and
transfer data relating to reprocessing cycles to the data management system.

3. System according to claim 1, wherein said RFID tag reader comprises adjustable guiding means constituted by an articulated arm adapted to direct a reading head of said RFID tag reader close to the RFID tags of the medical devices during reprocessing thereof.

4. System according to claim 1, further comprising a metal plate movably positioned outside the enclosure to prevent the RFID tag of the medical devices during reprocessing thereof from being read outside the enclosure.

5. System according to claim 1, wherein a plurality of said RFID tag readers are arranged to acquire identification information respectively from a plurality of said medical devices before, during and after the reprocessing cycles of said medical devices held in suspension inside the enclosure.

6. System according to claim 2, wherein the identification data comprise a unique identifier linked to each of the RFID tags of the medical devices.

7. System according to claim 2, wherein reprocessing cycle data include a number and type of reprocessing cycles performed, and error codes generated during reprocessing cycle execution.

8. System according to claim 1, wherein said RFID tag reader comprises guiding means arranged to guide a part of the RFID tag reader inside the enclosure so as to acquire identification data of each of the medical devices.

9. System for reprocessing medical devices according to claim 1, wherein said RFID tag reader is arranged completely in a wall of the enclosure so as to emit a signal and/or receive in return signals coming from the RFID tags of the medical devices suspended inside the enclosure.

10. System according to claim 1, wherein the RFID tag reader is formed by extrusion or overmolding in at least one wall of the enclosure.

11. System according to claim 1, wherein at least one said support has a tubular shape and at least one said ejector is arranged inside the tubular shape of said at least one support.

12. Method for reprocessing medical devices comprising the following steps:
i. equip a medical device to be reprocessed with an RFID tag;
ii. position inside an enclosure for reprocessing said medical device comprising said RFID tag and hold said medical device in suspension via a support;
iii. close the enclosure;
iv. adjust a position of said support in the enclosure and an orientation of said support relative to an RFID tag reader for said medical device to be reprocessed;
v. activate said RFID tag reader in order to collect identification data from the medical device before carrying out a reprocessing cycle;
vi. activate an ejector for reprocessing to perform at least one reprocessing cycle;
vii. collect data on reprocessing cycles;
viii. activate said RFID tag reader in order to collect identification data from the medical device after completion of the reprocessing cycles; and
ix. transfer the identification data acquired by said RFID tag reader and reprocessing cycle data to a data management system;
x. update data relating to said medical device in a database of said data management system after each reprocessing cycle.

13. Method for reprocessing medical devices according to claim 12, wherein the medical device to be reprocessed is placed inside an envelope held in suspension via said support inside the enclosure.

14. Method for reprocessing medical device according to claim 12, wherein said RFID tag reader is activated continuously throughout the reprocessing cycles.

15. Method for reprocessing medical devices according to claim 12, wherein the identification data for said medical device and reprocessing cycle data are transmitted by the electronic control system to the data management system so as to acquire traceability data for the medical device in real time.

16. Method for reprocessing medical devices according to claim 15, further comprising a supplementary validation step wherein the data management system is configured to validate the reprocessing of said medical device on the basis of said traceability data when no error code has been generated by said data management system.

17. Method for reprocessing of medical devices according to claim 12, said RFID tag reader comprising adjustable guiding means constituted by an articulated arm adapted to direct a reading head of said RFID tag reader in proximity to the RFID tag of the medical device, the method further comprising a step of adjusting a position of a part of said RFID tag reader inside the enclosure as a function of a type and size of said medical device to be reprocessed.
